(19) United States
(12) Patent Application Publication
(10) Pub. No.: US 2026/0299682 A1
(43) Pub. Date: Oct. 1, 2026

(54) PROCESSING DEVICE, EYE TRACKING SYSTEM AND PROCESSING METHOD

(71) Applicant: ams-OSRAM AG, Premstaetten (AT)

(72) Inventors: Volker ZAGOLLA, Prilly (CH); Ioannis PAPADOPOULOS, Lausanne (CH); Bastien MOYSSET, Martigny (CH); Jérôme MAYE, Martigny (CH)

(21) Appl. No.: 19/475,690

(22) PCT Filed: May 15, 2024
(86) PCT No.: PCT/EP2024/063375

§ 371 Date: Oct. 15, 2025

(30) Foreign Application Priority Data
May 30, 2023 (DE) 10 2023 114 150.3

(51) Int. Cl. G06F 3/01

(52) U.S. Cl. CPC G06F 3/013

(57) ABSTRACT

A processing device is configured to receive a first tracking signal from a first receiving unit, and to receive a second tracking signal from a second receiving unit, each of the first and the second tracking signals being a function of a speed of rotation and a position of a user's cornea. The processing device is further configured to determine the position of the user's cornea from the first and the second tracking signals.

The present disclosure relates to a processing device configured to determine the position of a user's cornea, an eye tracking system, and to a processing method.

Eye tracking is an important user side sensing modality that can enable a diverse set of applications e.g. in Augmented Reality (AR) applications.

Generally, concepts are sought by which the accuracy of detection may be improved.

It is an object of the present invention to provide an improved processing device, an improved eye tracking system and an improved processing method.

### SUMMARY

According to embodiments, the above objects are achieved by the claimed matter according to the independent claims. Further developments are defined in the dependent claims.

According to embodiments, a processing device is configured to receive a first tracking signal from a first receiving unit, and a second tracking signal from a second receiving unit, each of the first and the second tracking signals being a function of a speed of rotation and a position of a user's cornea. The processing device is further configured to determine the position of the user's cornea from the first and the second tracking signals.

For example, the processing device may be further configured to determine the speed of rotation of the user's cornea from the first and the second tracking signals.

For example, the processing device may be configured to determine a first plurality of candidate positions from the first tracking signal, to determine a second plurality of candidate positions from the second tracking signal and to select the candidate position that matches best to the first and the second tracking signals as the position of the user's cornea.

According to further embodiments, additionally, a plurality of candidate speeds may be determined from the first and the second tracking signals.

According to embodiments, the plurality of candidate positions may be determined using a neural network. For example, the neural network may be a convolutional and residual neural network.

According to further embodiments, the plurality of candidate positions may be determined using an analytical model.

For example, the first and the second tracking signals may be self-mixing interference (“SMI”) signals.

According to embodiments, the processing device may be configured to determine the first plurality of candidate positions from a frequency of the first tracking signal or from a frequency of a component of the first tracking signal. According to further embodiments, the first tracking signal may comprise signals having a plurality, e.g. 2 or 3 frequencies. In this case, one or the plurality of frequencies may be evaluated to determine the candidate positions and, optionally, speeds.

According to further embodiments, the processing device may be configured to further receive a third tracking signal from a third receiving unit. The third tracking signal may be a function of the speed of rotation and the position of the user's cornea. The processing device may be configured to determine the position of the user's cornea from the first, the second and the third tracking signals. According to further embodiments, the processing device may be configured to receive still further tracking signals from further receiving units. The further tracking signals may be functions of the speed of rotation and the position of the user's cornea. The processing device may be configured to determine the position of the user's cornea from the first, the second, the third and the further tracking signals. When more tracking signals are evaluated, the accuracy of determination may be improved.

According to embodiments, an eye tracking system comprises a first laser device configured to emit a first laser beam towards a user's cornea, and a second laser device configured to emit a second laser beam towards the user's cornea. The eye tracking system further comprises a first and a second receiving unit. The first receiving unit is configured to receive the first laser beam reflected by the cornea and to provide a first tracking signal and the second receiving unit is configured to receive the second laser beam reflected by the cornea and to provide a second tracking signal. The eye tracking system further comprises the processing device as described above.

For example, the first laser device may be configured to emit the first laser beam at a first fixed frequency.

Moreover, the second laser device may be configured to emit the second laser beam at a second fixed frequency.

According to embodiments, a method for determining a position of a user's cornea comprises receiving a first tracking signal from a first receiving unit, and receiving a second tracking signal from a second receiving unit, each of the first and the second tracking signals being a function of a speed of rotation and a position of a user's cornea. The method further comprises determining the position of the user's cornea from the first and the second tracking signals.

For example, the first laser beam may be emitted at a first fixed frequency. Further, the second laser beam may be emitted at a second fixed frequency.

For example, determining the position of the user's cornea may comprise determining a first plurality of candidate positions from the first tracking signal, determining a second plurality of candidate positions from the second tracking signal, and selecting the candidate position that matches best to the first and the second tracking signals as the position of the user's cornea.

Further embodiments relate to a computer program product comprising instructions, which, when the program is executed by a computer, cause the computer to carry out the method as explained above.

Still further embodiments relate to a computer-readable medium comprising instructions which, when executed by a computer, cause the computer to carry out the method as explained above.

### BRIEF DESCRIPTION OF THE DRAWINGS

The accompanying drawings are included to provide a further understanding of embodiments of the invention and are incorporated in and constitute a part of this specification. The drawings illustrate the embodiments of the present invention and together with the description serve to explain the principles. Other embodiments of the invention and many of the intended advantages will be readily appreciated, as they become better understood by reference to the following detailed description. The elements of the drawings are not necessarily to scale relative to each other. Like reference numbers designate corresponding similar parts.

FIG. 1A shows smart glasses comprising an eye tracking system according to embodiments.

FIG. 1B shows an enlarged schematical view of a laser output unit.

FIG. 2A illustrates illumination of the cornea using two laser devices.

FIG. 2B illustrates illumination of cornea when the eye rotates.

FIG. 3 illustrates processing in the processing device.

FIGS. 4A, 4B, 4C, 4D and 4E further illustrate processing in the processing device.

FIG. 5 summarizes a method according to embodiments.

### DETAILED DESCRIPTION

In the following detailed description reference is made to the accompanying drawings, which form a part hereof and in which are illustrated by way of illustration specific embodiments in which the invention may be practiced. In this regard, directional terminology such as “top”, “bottom”, “front”, “back”, “over”, “on”, “above”, “leading”, “trailing” etc. is used with reference to the orientation of the Figures being described. Since components of embodiments of the invention can be positioned in a number of different orientations, the directional terminology is used for purposes of illustration and is in no way limiting. It is to be understood that other embodiments may be utilized and structural or logical changes may be made without departing from the scope defined by the claims.

The description of the embodiments is not limiting. In particular, elements of the embodiments described hereinafter may be combined with elements of different embodiments.

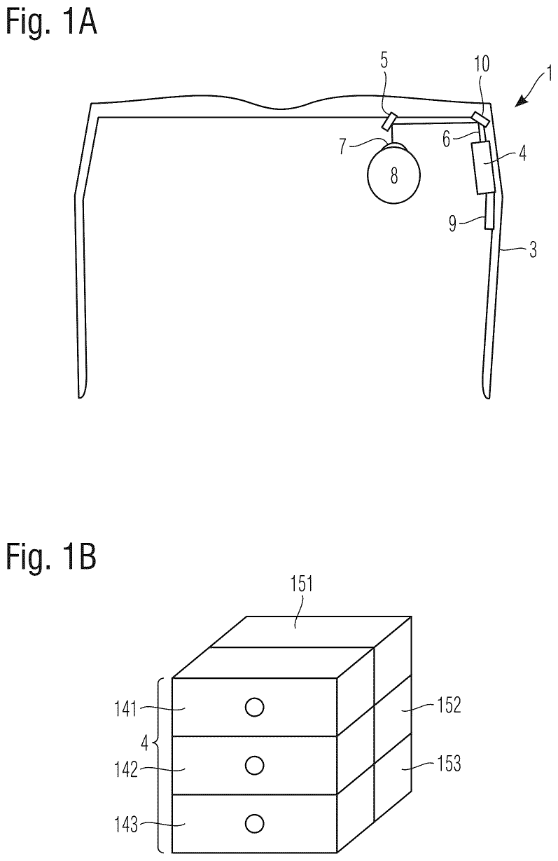

FIG. 1A shows an eye tracking system 1 that may be integrated in a pair of glasses (e.g. AR/VR smart glasses or a headset or headup display) comprising a frame 2 having a stem 3. The eye tracking system 1 comprises a laser output unit 4. The laser output unit 4 may comprise a plurality of laser devices, as will be explained below with reference to FIG. 1B. The eye tracking system 1 may further comprise an optical element 5 for modulating laser beams 6 from the laser output unit 4. According to implementations, the optical element may be transmissive, reflective or diffractive. The optical element 5 may be implemented as a holographic optical element, e.g. a volume phase hologram. The optical element 5 may further be focusing and/or collimating. Depending on the location of the laser output unit, the optical element 5 may be dispensed with. For example, the optical element 5 may provide a plane wave illumination field on the cornea 7 of the eye 8 of the user/wearer of the glasses. A part of the laser beams 6 is reflected back from the cornea 7. The reflected beams may be directed into respective receiving units. For example, the receiving units may be integrated in the laser output unit 4.

For example, the reflected light may interfere with the laser field in the laser devices through so called self mixing interference, which changes the output of the laser devices.

The system 1 comprises a processing device 9 for determining the distance to the cornea 7 based on self mixing interferometry (SMI). As the user's eye 8 moves the distance to the reflection point on the cornea 7 shifts, which causes the distance between the laser source and the cornea 7 to change. Hence, the eye movement can be detected. According to embodiments, a frequency of the laser beams 6 generated by the laser output unit 4 does not change and may be constant. As a result, the signal reflected by the cornea 7 only depends on the movement or rotation of the cornea. For example, it is possible that the signal reflected by the cornea 7 does not depend from the distance between the cornea 7 and the receiving units 151, 152.

To accurately determine the rotation of the eye in all directions, at least two angularly and/or spatially separated laser beams 6 are used. More beams may be used for more precise eye tracking and/or to provide redundancy in the system. Reducing the number of laser beams 6 results in a reduced power consumption and a reduction of the form factor. The eye tracking system 1 may further comprise a second optical element 10 being a mirror for redirecting the laser beams 6 from the laser output unit 4. Further optical elements can provide greater flexibility in the placement of the laser output unit 4.

According to further embodiments, the laser output unit 4 may be placed in the frame in front of the eye 8 and transmit the laser beams 6 directly onto the cornea 7, without requiring a reflective element. Also, it is not necessary to place the laser output unit 4 in the glasses. Instead the laser output unit 4 may be placed off the glasses and be connected to waveguides for transmitting the laser beams 6.

Further, the processing device 9 may be arranged outside the glasses. For example, the processing device 9 may be placed in or may be a component of a computer or an electronic device such as a mobile electronic user device or smartphone and may be configured to receive signals from the receiving units.

The eye-tracking system 1 comprises a small number of illumination and detection devices to measure the relative depth change of specific points on the cornea 7 and use this information to infer the gaze direction of the eye 8. The SMI or tracking signals used to detect the depth change of each point can be measured, e.g. using an integrated photodiode in the laser source, or via direct measurement of the voltage applied to the laser devices. The use of laser illumination and detection enables the combination of the sensing module with highly transparent and efficient reflective or diffractive optical components placed in the eyepiece. It is exactly this that allows the flexible placement of the sensing module within the stem 3 of head wearable devices. The combination of SMI detection (capable of measuring very small depth changes of the point of interest) together with the placement of the various illumination and detection sources in a particular geometry enables very high accuracy over the full eye rotation space. Finally, the solution comprising of a minimum of two sources leads to an overall low power consumption.

FIG. 1B is a schematic view of a laser output unit 4 according to embodiments. The laser output unit 4 comprises a plurality of laser devices, e.g. a first laser device 141, a second laser device 142, and, optionally, a third laser device 143. For example, each of the laser devices may be implemented as a surface emitting laser, e.g. a VCSEL (“vertical cavity surface emitting laser”). The laser devices 141, 142, 143 are arranged at different positions. For example, the laser devices may be arranged in one direction, e.g. a horizontal or a vertical direction. According to further embodiments, the laser devices may be arranged in a two-dimensional pattern, e.g. in the shape of a triangle or a rectangle.

The laser output unit 4 further comprises a plurality of receiving units, e.g. a first receiving unit 151, a second receiving unit 152, and, optionally, a third receiving unit 153. The first receiving unit 151 may be configured to detect a first tracking or SMI signal that is based on the superposition of a first laser beam emitted by the first laser device 141 and the first laser beam that has been reflected by the cornea. In a similar manner, the second receiving unit 152 may be configured to detect a second tracking or SMI signal that is based on the superposition of a second laser beam emitted by the second laser device 142 and the second laser beam that has been reflected by the cornea.

The receiving units 151, 152, 153 may be implemented as separate photodetectors or may be integrated in the laser devices 141, 142 or 143, e.g. measuring the input voltage or input current to the laser devices.

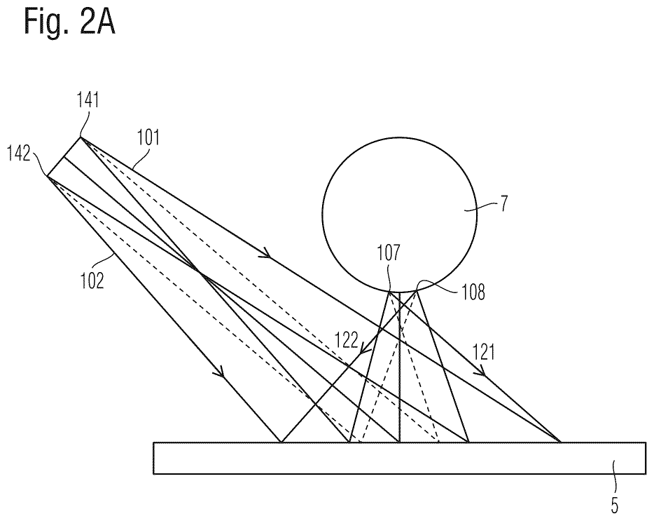

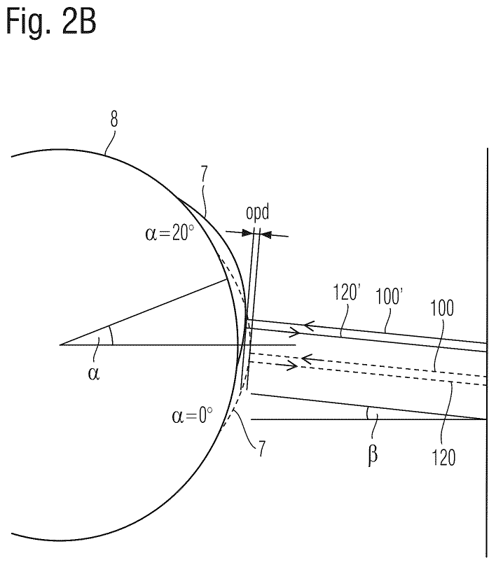

FIG. 2A shows an illumination scheme using a first laser device 141 and a second laser device 142 and a first optical element 5 which is implemented as a diffractive optical element. The first and the second laser devices 141, 142 may be implemented as VCSELs. The first measurement point 107 refers to a first laser beam 101 emitted by the first laser device 141, the second measurement point 108 refers to a second laser beam 102 emitted by the first laser device 142. Due to the center configuration of the laser devices 141, 142 in regard to the optical axis of the first optical element 5, the first measurement point 107 (corresponding to the focal point of the first optical element 5) on the cornea 7 is spatially different from the second measurement point 108. As is clearly to be understood, more than two laser devices may be employed.

FIG. 2B shows how rotation of the eye 8 changes the optical path when using plane wave illumination, resulting in a measurable signal. The cornea 7 of the eye 8 may be modeled as a sphere. When the eye 8 rotates, the center of rotation may be assumed to be the center of the eyeball. Therefore, the rotation of the eye 8 will lead to a displacement of the cornea.

A laser beam 100 having an illumination angle β is incident on and reflected from a point on the cornea 7 of the eye 8 when the eye 8 is in a first position (eye rotation α=0°). Based on the input angle β, there is one point on the cornea 7 that, due to the local curvature, will reflect light at the same angle as the incident angle and back (reflected beam 120) to the receiving unit (not shown). This point remains the same relative to the center of the cornea 7 when the eye 8 rotates. The eye 8 is rotated into a second position (α=20°), in which the laser beam 100′ is incident on and reflected from a point on the cornea 7 and back (reflected beam 120′) to the receiving unit. The change in distance of the optical path (the optical path difference, OPD) of the reflected laser beams 120 and 120′ between the cornea 7 and the receiving unit depends on the angle of rotation α and can therefore be measured to determine the eye rotation.

In more detail, a frequency of fringes of a tracking or SMI signal 111, 112, 113 measured by the receiving unit depends both on the speed v of rotation and the actual position θ of the cornea 7 with respect to the laser beam.

In other words, the following relationship holds:

$$fringe frequency = f ( θ , v ) .$$

In terms of information, the above implies that having 2 (or possibly more) independent measurements of the local fringe frequency of the eye rotation may enable the prediction of both angular position θ and speed v by solving the set of above equations. In this context, the set of equations may refer to the measurements performed by the plurality of receiving units. According to embodiments, the equations may be solved utilizing predictors, optimization algorithms, machine learning, neural networks and others.

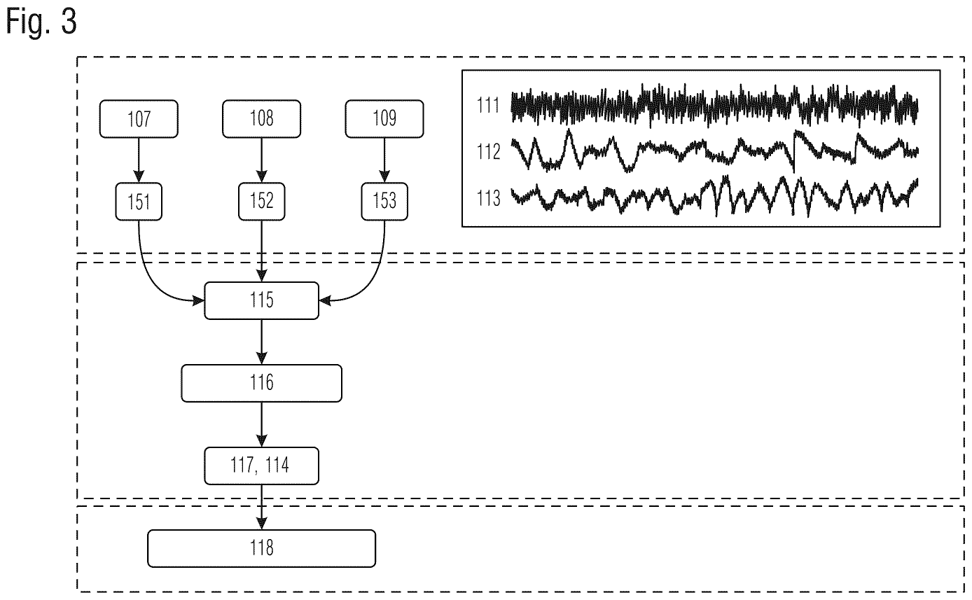

FIG. 3 illustrates stages of processing that may be performed in the processing device 9. A first laser device (not shown in FIG. 3) is configured to emit a first laser beam towards a user's cornea. The point of reflection at the cornea corresponds to a first measurement point 107. A second laser device (not shown in FIG. 3) is configured to emit a second laser beam towards the user's cornea. The point of reflection at the cornea corresponds to a second measurement point 108. Optionally, a third laser device (not shown in FIG. 3) is configured to emit a third laser beam towards a user's cornea. The point of reflection at the cornea corresponds to a third measurement point 109. The used laser devices have a fixed relative position with respect to each other. Further, the optical system between the laser devices and the eyes is fixed.

A first tracking signal 111 which may be generated by interference of the first laser beam and the first reflected beam (not illustrated in FIG. 3) is received by the first receiving unit 151. A second tracking signal 112 which may be generated by interference of the second laser beam and the second reflected beam (not illustrated in FIG. 3) is received by the second receiving unit 152. Optionally, a third tracking signal 113 which may be generated by interference of the third laser beam and the third reflected beam (not illustrated in FIG. 3) is received by the third receiving unit 153. The inset on the right-hand side of FIG. 3 shows examples of the first, second and third tracking signals 111, 112, 113.

In stage 115, the tracking signals 111, 112, 113 are combined and may be treated to bring them into a comparable level. For example, the data may be normalized and down-sampled. In stage 116, a signal preprocessing may be performed. Then, the data are processed. For example, a neural network model 117 or an analytical model 114 may be applied to the data. As a result, the absolute position 118 of the eye 8 may be determined.

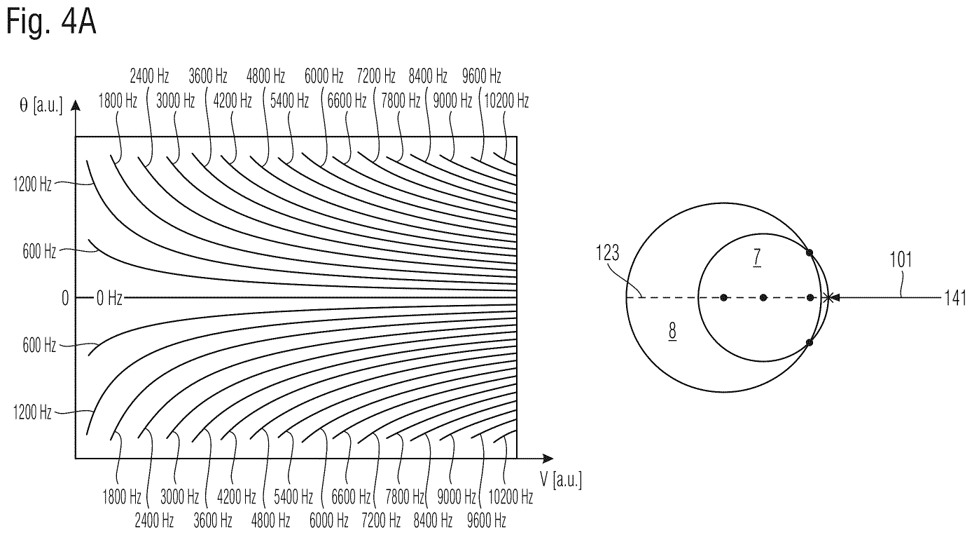

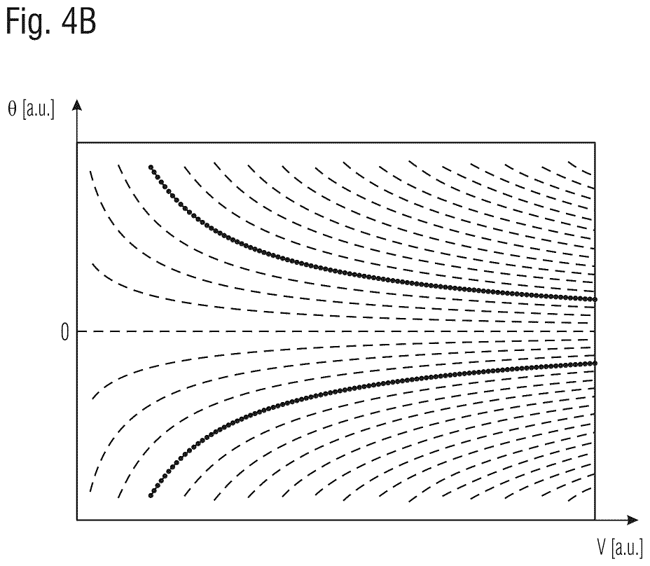

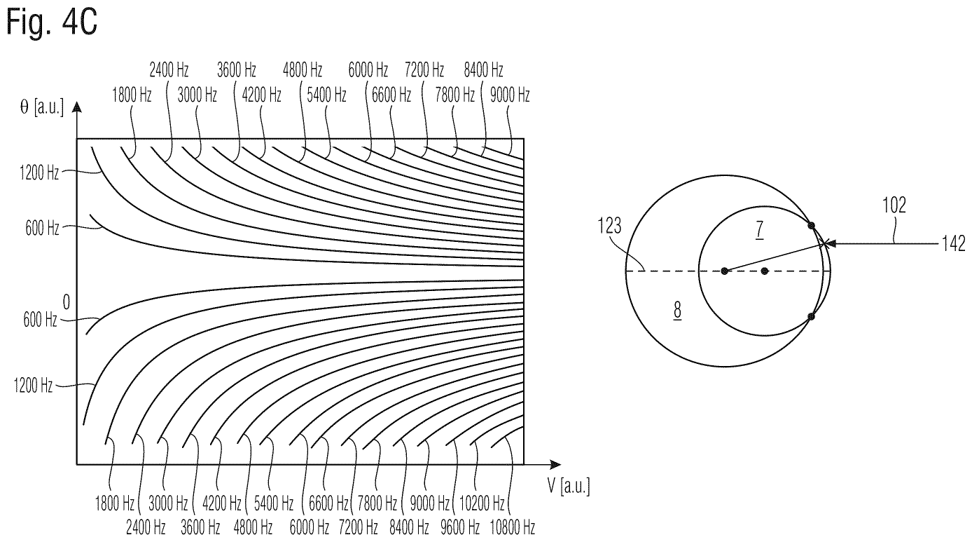

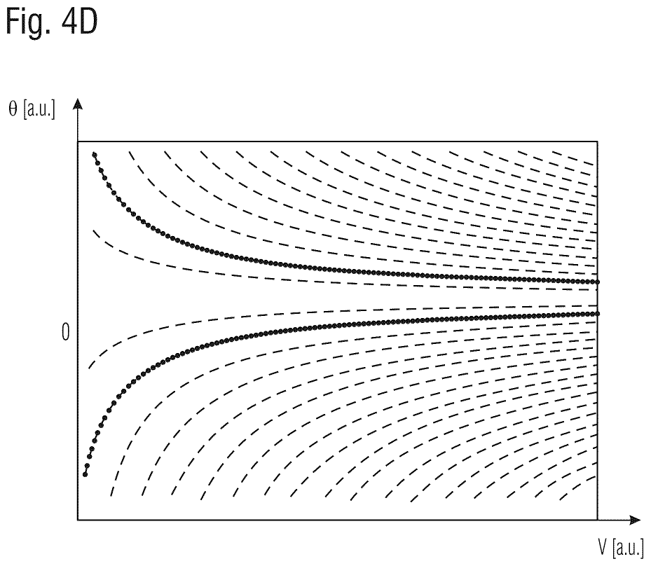

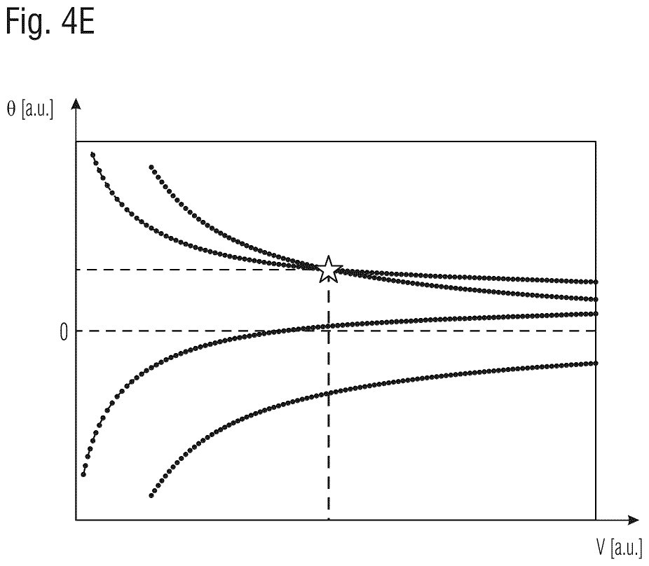

An example of determining the absolute position of the eye 8 is described below while referring to FIGS. 4A to 4E.

As has been explained above with reference to FIG. 2B the SMI signal (or fringe) frequency of the tracking signals depends on both angular position θ and speed v of rotation. Accordingly, there exist infinite combinations of angular position and speed v of rotation for a given SMI signal (or fringe) frequency. According to embodiments, an analytical model may be used. For example, the analytical model may be based on trigonometrics. As is clearly to be understood, any other model may be employed.

For example, further models may rely on neural networks, e.g. convolutional neural networks and others.

FIG. 4A illustrates a plurality of possible solutions for speed v of rotation and angular position θ when the laser device and receiving unit are aligned with an axis of symmetry 123 of the eye 8 and not shifted down- or upwards. These possible solutions include a plurality of candidate positions for different fringe frequencies. The right-hand portion of FIG. 4A illustrates the position of the first laser device 141 and the first laser beam 101 with respect to the axis of symmetry 123 of the eye 8.

It is assumed that the first tracking signal 111 measured by the first receiving unit 151 has a local SMI signal or fringe frequency of 2400 Hz.

Possible solutions including a plurality of candidate positions for a fringe frequency of 1200 Hz are highlighted in FIG. 4B. Further, FIG. 4B shows in broken lines possible solutions or combinations of angular position and speed of rotation for a plurality of fringe frequencies different from 2400 Hz.

The second laser device 142 and the second receiving unit 152 are shifted with respect to the first laser device 141 and the first receiving unit 151. Accordingly, the second laser device 142 and the second receiving unit 152 are not aligned with the axis of symmetry 123 of the eye 8. As a result, the fringe frequency of the second tracking signal 112 received by the second receiving unit 152 is shifted along the angular position axis. This is illustrated in FIG. 4C. The right-hand portion of FIG. 4C illustrates the position of the second laser device 142 and the second laser beam 102 with respect to the axis of symmetry 123 of the eye 8.

It is further assumed that the second tracking signal 112 measured by the second receiving unit 152 has a local SMI signal or fringe frequency of 1200 Hz. Possible solutions including a plurality of candidate positions for a fringe frequency of 1200 Hz are highlighted in FIG. 4D. Further, FIG. 4D shows in broken lines possible solutions or combinations of angular position and speed of rotation for a plurality of fringe frequencies different from 1200 Hz.

As is illustrated in FIG. 4E, by superimposing the candidate solutions of FIGS. 4B and 4D, the solution that matches both measurements may be determined. In other words, the measurements from two different viewpoints intersect at one single point in the e, v-space. This single point is the position and speed determined from the two measurements.

The processing method may be modified by receiving a third or still further tracking signals. By evaluating further tracking signals, the accuracy may be improved.

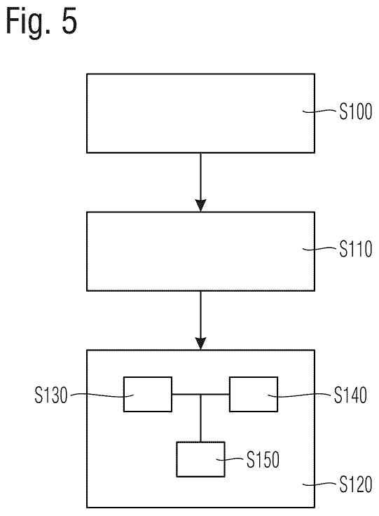

FIG. 5 summarizes a method according to embodiments. As is shown, a method for determining a position of a user's cornea comprises receiving (S100) a first tracking signal from a first receiving unit, and receiving (S110) a second tracking signal from a second receiving unit, each of the first and the second tracking signals being a function of a speed of rotation and a position of a user's cornea. The method further comprises determining (S120) the position of the user's cornea from the first and the second tracking signals. For example, determining (S120) the position of the user's cornea may comprise determining (S130) a first plurality of candidate positions from the first tracking signal, determining (S140) a second plurality of candidate positions from the second tracking signal, and selecting (S150) the candidate position that matches best to the first and the second tracking signals as the position of the user's cornea. According to embodiments, the first plurality of candidate positions may be determined (S130) before, after or at the same time as determining (S140) the second plurality of candidate positions.

As has been described above, the absolute position of the cornea or the eye may be tracked. In contrast to methods in which a relative movement is determined and the absolute position may be determined in dependence from a starting position, the method described is less susceptible to error, when there is some measurement error. In particular, accumulated errors may be reduced or suppressed.

The method described above may be performed by the processing device 9. For example, the processing device may be a component of an eye tracking system 1 as has been explained above. According to embodiments, the processing device 9 may be remote from the laser output unit 4. For example, the processing device may be implemented as a computer or an electronic user device. According to further embodiments, the processing device may be a component of a computer, or an electronic user device. In these cases, the processing device 9 may be operable to receive the tracking signals from the first and the second receiving units 151, 152.

Further embodiments relate to a computer program product comprising instructions, which, when the program is executed by a computer, cause the computer to carry out the method as described above.

Still further embodiments relate to a computer-readable medium comprising instructions which, when executed by a computer, cause the computer to carry out the method as described above.

While embodiments of the invention have been described above, it is obvious that further embodiments may be implemented. For example, further embodiments may comprise any subcombination of features recited in the claims or any subcombination of elements described in the examples given above. Accordingly, this spirit and scope of the appended claims should not be limited to the description of the embodiments contained herein.

### LIST OF REFERENCES

1 eye tracking system
2 frame
3 stem
4 laser output unit
5 first optical element
6 laser beam
7 cornea
8 eye
9 processing device
10 second optical element
100, 100′ laser beam
101 first laser beam
102 second laser beam
107 first measurement point
108 second measurement point
109 third measurement point
111 first tracking signal
112 second tracking signal
113 third tracking signal
114 analytical model
115 combined data
116 signal preprocessing
117 neural network model
118 absolute position
120, 120′ reflected beam
121 first reflected beam
122 second reflected beam
123 axis of symmetry
141 first laser device
142 second laser device
143 third laser device
151 first receiving unit
152 second receiving unit
153 third receiving unit

1. A processing device configured
to receive a first tracking signal from a first receiving unit,
to receive a second tracking signal from a second receiving unit, each of the first and the second tracking signals being a function of a speed of rotation and a position of a user's cornea,
the processing device being further configured to determine the position of the user's cornea from the first and the second tracking signals.

2. The processing device according to claim 1, being further configured to determine the speed of rotation of the user's cornea from the first and the second tracking signals.

3. The processing device according to claim 1, which is configured to determine a first plurality of candidate positions from the first tracking signal, to determine a second plurality of candidate positions from the second tracking signal and to select the candidate position that matches best to the first and the second tracking signals as the position of the user's cornea.

4. The processing device according to claim 3, which is configured to determine the first plurality of candidate positions from a frequency of the first tracking signal.

5. The processing device according to claim 3, wherein the plurality of candidate positions are determined using a neural network.

6. The processing device according to claim 5, wherein the neural network is a convolutional and residual neural network.

7. The processing device according to claim 2, wherein the plurality of candidate positions are determined using an analytical model.

8. The processing device according to claim 1, wherein the first and the second tracking signals are self-mixing interference signals.

9. The processing device according to claim 1, being configured to further receive a third tracking signal from a third receiving unit,
the third tracking signal being a function of the speed of rotation and the position of the user's cornea,
the processing device being configured to determine the position of the user's cornea from the first, the second and the third tracking signals.

10. An eye tracking system, comprising:
a first laser device configured to emit a first laser beam towards a user's cornea,
a second laser device configured to emit a second laser beam towards the user's cornea,
a first and a second receiving unit, wherein the first receiving unit is configured to receive the first laser beam reflected by the cornea and to provide a first tracking signal and the second receiving unit is configured to receive the second laser beam reflected by the cornea and to provide a second tracking signal; and
a processing device configured
to receive a first tracking signal from a first receiving unit,
to receive a second tracking signal from a second receiving unit, each of the first and the second tracking signals being a function of a speed of rotation and a position of a user's cornea,
the processing device being further configured to determine the position of the user's cornea from the first and the second tracking signals.

11. The system according to claim 10, wherein the first laser device is configured to emit the first laser beam at a first fixed frequency.

12. The system according to claim 10, wherein the second laser device is configured to emit the second laser beam at a second fixed frequency.

13. A method for determining a position of a user's cornea comprising:
receiving a first tracking signal from a first receiving unit, the first tracking signal being based on a first laser beam that has been emitted towards the cornea and that has been reflected by the cornea,
receiving a second tracking signal from a second receiving unit, the second tracking signal being based on a second laser beam that has been emitted towards the cornea and that has been reflected by the cornea,
each of the first and the second tracking signals being a function of a speed of rotation and a position of the user's cornea, and
determining the position of the user's cornea from the first and the second tracking signals.

14. The method according to claim 13, wherein the first laser beam is emitted at a first fixed frequency.

15. The method according to claim 13, wherein the second laser beam is emitted at a second fixed frequency.

16. The method according to claim 13, wherein determining the position of the user's cornea comprises
determining a first plurality of candidate positions from the first tracking signal,
determining a second plurality of candidate positions from the second tracking signal, and
selecting the candidate position that matches best to the first and the second tracking signals as the position of the user's cornea.

17. (canceled)

18. (canceled)
